(19) United States
(12) Patent Application Publication
(10) Pub. No.: US 2023/0357433 A1
(43) Pub. Date: Nov. 9, 2023

(54) INFLAMMATORY CYTOKINES AND FATIGUE IN SUBJECT WITH A COMPLEMENT MEDIATED DISEASE

(71) Applicant: Bioverativ USA Inc., Waltham, MA (US)

(72) Inventors: Jaime Morales Arias, Waltham, MA (US); William E. Hobbs, Waltham, MA (US); Michael J. Storek, Cambridge, MA (US); Parija S. Patel, Waltham, MA (US)

(73) Assignee: Bioverativ USA Inc., Waltham, MA (US)

(21) Appl. No.: 18/164,521

(22) Filed: Feb. 3, 2023

Related U.S. Application Data
(63) Continuation of application No. PCT/US2021/044761, filed on Aug. 5, 2021.
(60) Provisional application No. 63/062,243, filed on Aug. 6, 2020.

(51) Int. Cl. C07K 16/40; G01N 33/68; A61P 7/00

(52) U.S. Cl. CPC C07K 16/40; G01N 33/6893; A61P 7/00; G01N 2800/224; G01N 2333/5412; G01N 2800/306; C07K 2317/52; G01N 2333/5428; G01N 2800/52

(57) ABSTRACT

Provided herein are methods for treating complement-mediated diseases and associated conditions.

### RELATED APPLICATION

This application claims priority to U.S. provisional application Ser. No. 63/062,243, filed Aug. 6, 2020, the entire content of which is incorporated herein by reference.

### FIELD

The present application relates to methods for treating complement-mediated diseases and associated conditions.

### BACKGROUND

Cold agglutinin disease (CAD) is a rare, chronic type of autoimmune hemolytic anemia, where hemolysis is driven by classical complement pathway activation. Complement activation assures the rapid initiation of the complement cascade as a part of an early immune response.

### SUMMARY

A pro-inflammatory state secondary to complement activation has been demonstrated in other hemolytic diseases such as paroxysmal nocturnal hemoglobinuria and atypical-hemolytic uremic syndrome. Complement cascade activation stimulates cytokine production (TNF, IL-6, IL-8, IL-17) via anaphylatoxins C3a and C5a and increases vascular inflammatory markers, consistent with complement-mediated inflammation. In addition, complement activation and chronic inflammation may contribute to patient fatigue in cold agglutinin disease (CAD), apart from anemia.

Classical complement activation resulting in a proinflammatory state has not been formally studied in CAD patients. Also, the interplay between complement-mediated inflammation, and fatigue has not been explored in patients with CAD. Results from the study described herein demonstrate a relationship between inflammatory cytokine expression (e.g., IL-6 and IL-10) and fatigue in CAD patients treated with the humanized monoclonal anti-C1s antibody, sutimlimab.

Some aspects of the present disclosure provide a method comprising administering to a subject an anti-C1s antibody (e.g., the anti-C1s antibody of Table 1 or Table 2) and measuring a level of C reactive protein (CRP), IL-6, and/or IL-10 in a sample (e.g., blood, e.g., serum) from the subject. In some embodiments, the method further comprises assessing fatigue (e.g., measuring FACIT-F score) in the subject.

Other aspects of the present disclosure provide a method comprising measuring a level of CRP, IL-6, and/or IL-10 in a sample from a subject being treated with an anti-C1s antibody (e.g., sutimlimab). In some embodiments, the method further comprises assessing fatigue in the subject.

In some embodiments, a level of CRP is measured. CRP, in some embodiments, is used as a surrogate for IL-6. In some embodiments, a level of IL-6 is measured. In some embodiments, IL-10 is measured.

In some embodiments, the subject has a complement-mediated disease. In some embodiments, the subject has CAD.

In some embodiments, the subject has fatigue.

Yet other aspects of the present disclosure provide a method comprising treating a subject with an anti-C1s antibody therapy (e.g., sutimlimab therapy), wherein the subject has fatigue, and measuring a level of CRP, IL-6, and/or IL-10 in a sample from the subject. In some embodiments, the method further comprises assessing fatigue in the subject. In some embodiments, the subject has a complement-mediated disease, for example, CAD.

Further aspects of the present disclosure provide a method comprising treating a subject with an anti-C1s antibody therapy (e.g., sutimlimab therapy), wherein the subject has a complement mediated disease, for example, CAD, and measuring a level of CRP, IL-6, and/or IL-10 in a sample from the subject. In some embodiments, the method further comprises assessing fatigue in the subject. In some embodiments, the subject has fatigue.

In some embodiments, the subject has a baseline level of CRP, IL-6, and/or IL-10 prior to treatment with an anti-C1s antibody (e.g., sutimlimab), and/or wherein the subject has a baseline level of fatigue prior to treatment with an anti-C1s antibody. In some embodiments, if the level of CRP, IL-6, and/or IL-10 in the sample is reduced, for example, by at least 5% or at least 10% (e.g., at least 15%, 20%, 25%, 30%, 35%, 40%, 45%, or 50%), relative to baseline and/or fatigue in the subject is improved relative to baseline, the method further comprises continuing with a current anti-C1s antibody treatment (e.g., sutimlimab treatment). In some embodiments, if the level of CRP, IL-6, and/or IL-10 in the sample is within 5% or within 10% of baseline and/or fatigue the subject is maintained or worsens relative to baseline, the method further comprises altering a current anti-C1s antibody treatment. In some embodiments, if the level of CRP, IL-6, and/or IL-10 in the sample is within 5% or within 10% of baseline, the method further comprises altering a current anti-C1s antibody treatment. In some embodiments, if fatigue the subject is maintained or worsens relative to baseline, the method further comprises altering a current anti-C1s antibody treatment.

In some embodiments, altering the current anti-C1s antibody treatment comprises adjusting the dosage and/or frequency of the treatment with the anti-C1s antibody.

In some embodiments, altering the current anti-C1s antibody treatment comprises further treatment of the subject with an anti-inflammatory agent.

In some embodiments, altering the current anti-C1s antibody treatment comprises further treatment of the subject to improve fatigue.

In some embodiments, a method further comprises monitoring levels (e.g., reassessing levels) of CRP, IL-6, and/or IL-10 in the subject over a period of time (e.g., hours, days, weeks, or months).

In some embodiments, the subject has undergone a blood transfusion. In some embodiments, the subject has undergone a blood transfusion prior to commencing treatment with an anti-C1s antibody, e.g., within a month, within 3 weeks, within 2 weeks, or within 1 week.

In some embodiments, the fatigue is assessed based on a Functional Assessment of Chronic Illness Therapy-Fatigue (FACIT-F) score. In some embodiments, an improvement in fatigue is a change by at least 3 (e.g., at least 4, 5, 6, 7, 8, 9, or 10) points on a FACIT-F score relative to baseline.

In some embodiments, the anti-C1s antibody comprises a heavy chain (HC) complementarity determining region 1 (CDR1) comprising the amino acid sequence of SEQ ID NO: 5, an HC complementarity determining region 2 (CDR2) comprising the amino acid sequence of SEQ ID NO: 6, an HC complementarity determining region 3 (CDR3) comprising the amino acid sequence of SEQ ID NO: 7, a light chain (LC) CDR1 that comprises the amino acid sequence of SEQ ID NO: 8, an LC CDR2 comprising the amino acid sequence of SEQ ID NO: 9, and an LC CDR3 comprising the amino acid sequence of SEQ ID NO: 10.

In some embodiments, the anti-C1s antibody comprises a heavy chain variable region (VH) comprising the amino acid sequence of SEQ ID NO: 3 and comprises a light chain variable region (VL) comprising the amino acid sequence of SEQ ID NO: 4.

In some embodiments, the anti-C1s antibody comprises an HC comprising the amino acid sequence of SEQ ID NO: 1 and an LC comprising the amino acid sequence of SEQ ID NO: 2.

In some embodiments, the anti-C1s antibody comprises an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, an LC CDR1 comprising the amino acid sequence of SEQ ID NO: 18, an LC CDR2 comprising the amino acid sequence of SEQ ID NO: 19, and an LC CDR3 comprising the amino acid sequence of SEQ ID NO: 20.

In some embodiments, the anti-C1s antibody comprises a VH comprising the amino acid sequence of SEQ ID NO: 13 and comprises a VL comprising the amino acid sequence of SEQ ID NO: 14.

In some embodiments, the anti-C1s antibody comprises an HC comprising the amino acid sequence of SEQ ID NO: 11 and an LC comprising the amino acid sequence of SEQ ID NO: 12.

In some embodiments, the anti-C1s antibody comprises an IgG4 constant region. Each of International Publication Nos. WO 2014/071206, filed Nov. 2, 2012, entitled Anti-Complement C1s Antibodies and Uses Thereof, WO 2016/164358, filed Apr. 6, 2015, entitled Humanized Anti-C1s Antibodies and Methods of Use Thereof, and in WO 2018/170145, filed Mar. 14, 2017, entitled Methods for Treating Complement-Mediated Diseases and Disorders is herein incorporated by reference in its entirety.

### BRIEF DESCRIPTION OF THE DRAWINGS

FIG. 1 shows changes in proinflammatory cytokine IL-6 levels (B) and FACIT-F (A) scores at baseline and over time in patients with cold agglutinin disease (CAD). FACIT-F, functional assessment of chronic illness therapy-fatigue; SEM, standard error of the mean; TAT, treatment assessment timepoint. Mean and SEM values from week 25 were used to represent the TAT. Normal control values for IL-6 were <3.2 pg/mL.

FIG. 2 shows changes in regulatory cytokine IL-10 levels (B) and FACIT-F (A) scores at baseline and over time in patients with CAD. FACIT-F, functional assessment of chronic illness therapy-fatigue; SEM, standard error of the mean; TAT, treatment assessment timepoint. Mean and SEM values from week 25 were used to represent the TAT.

FIG. 3 shows effect of sutimlimab treatment on mean IL-6 level (A), fatigue (B), mean total C4 (C), and classical complement pathway activity (D) in patients with CAD. Mean and SEM values from Week 25 were used to represent the TAT. Normal values for IL-6 were <3.2 pg/mL. CAD, cold agglutinin disease; CP, classical complement pathway; FACIT-Fatigue, Functional Assessment of Chronic Illness Therapy-Fatigue; IL, interleukin; SEM, standard error of the mean; TAT, treatment assessment time point.

FIG. 4 shows effect of sutimlimab treatment on mean IL-10 level (A), fatigue (B), mean total C4 (C), and classical complement pathway activity (D) in patients with CAD. Mean and SEM values from Week 25 were used to represent the TAT. CAD, cold agglutinin disease; CP, classical complement pathway; FACIT-Fatigue, Functional Assessment of Chronic Illness Therapy-Fatigue; IL, interleukin; SEM, standard error of the mean; TAT, treatment assessment time point.

### DESCRIPTION

The complement system is a well-known effector mechanism of the immune response, providing not only protection against pathogens and other harmful agents but also recovery from injury. The complement pathway comprises proteins that typically exist in the body in inactive form. The classical complement pathway is triggered by activation of the first component of complement, referred to as the C1 complex, which includes C1q, C1r, and C1s proteins. Upon binding of C1 to an immune complex or other activator, the C1s component, a diisopropyl fluorophosphate (DFP)-sensitive serine protease, cleaves complement components C4 and C2 to initiate activation of the classical complement pathway. The classical complement pathway plays a role in cold agglutinin disease, for example.

Cold agglutinin disease (CAD) is a form of chronic autoimmune hemolytic anemia (AIHA) accompanied by classical component-dependent hemolysis (see Berentsen S. Semin Hematol. 2018; 55(3):141-149; and Noris M, Remuzzi G. Semin Nephrol. 2013; 33(6):479-492, each of which is herein incorporated by reference). Symptoms of CAD can include chronic hemolysis, anemia and related symptoms (e.g., dyspnea), hemoglobinuria, jaundice, and circulatory symptoms. Some people who have cold agglutinin disease also may get a cold, numb feeling and loss of color in their fingers or toes, known as Raynaud's phenomenon.

The data provided herein demonstrated a relationship between inflammatory cytokine expression (e.g., IL-6 and IL-10) and fatigue in CAD patients treated with the humanized monoclonal anti-C1s antibody, sutimlimab.

IL-6 is a pro-inflammatory cytokine featuring pleiotropic activity; it induces synthesis of acute phase proteins such as CRP, serum amyloid A, fibrinogen, and hepcidin in hepatocytes, whereas it inhibits production of albumin. IL-6 also plays an important role on acquired immune response by stimulation of antibody production and of effector T-cell development. Moreover, IL-6 can promote differentiation or proliferation of several nonimmune cells. Because of the pleiotropic activity, dysregulated continual production of IL-6 leads to the onset or development of various diseases.

IL-10, formerly known as cytokine synthesis inhibitory factor and namesake of the corresponding cytokine family, is regarded as a key immunoregulatory cytokine capable of curbing overt inflammation in various pathophysiological settings. In addition to influencing the cytokine network, IL-10 has the capability to curb production of key effector mediators involved in development of tissue damage, among others reactive oxygen species (and matrix metalloproteinases.

Fatigue is a common symptom of CAD. While not solely caused by anemia, the fatigue in CAD is presumed to be secondary to the hemolytic anemia, which refers to low numbers of RBCs as a result of their destruction. RBCs are responsible for delivering oxygen to all parts of the body, and for removing carbon dioxide that is generated because of metabolic activity. When RBC numbers are low due to hemolytic anemia, the organs receive less oxygen than required for their normal function. This affects all organs of the body, especially those that require high energy for their function. The result is tiredness and fatigue because the body is not able to keep up with the energy demand of daily tasks.

### Methods of Monitoring Inflammation and Fatigue

The present disclosure is based in part, on the observation that subjects with a complement-mediated disease (e.g., CAD), who are receiving treatment with an anti-C1s antibody, show a decrease in the levels of inflammatory cytokines along with a concomitant improvement in symptoms of fatigue.

Accordingly, the present disclosure provides methods of monitoring and/or treating inflammation and/or fatigue in subjects with complement-mediated disorders, by analyzing the status of one or more inflammatory cytokines (e.g., IL-6, IL-8, IL-10, IL-12, IL-17, IL-18, IL-1β, IFN-γ, TNF-α, TNF-receptor, etc.) in said patients. In some embodiments, the status of one or more inflammatory cytokines is determined by measuring the level of one or more biomarkers of inflammation. In some embodiments, the levels of inflammatory cytokines and/or biomarkers of inflammation provide an objective measure of fatigue. Methods of measuring levels of inflammatory cytokines and/or biomarkers of inflammation are known in the art. The level of an inflammatory cytokine or biomarker of inflammation may be measured using standard electrophoretic and immunodiagnostic techniques, including but not limited to immunoassays such as competition, direct reaction, or sandwich type assays. Such techniques include, but are not limited to, Western blots; agglutination tests; enzyme-labeled and mediated immunoassays, such as an ELISA; biotin/avidin assays; radioimmunoassays; immunoelectrophoresis; immunoprecipitation, etc. The methods may include plasmon resonance methods, or any method that detects the presence of an inflammatory cytokine or biomarker of inflammation by the binding of an antibody, aptamer, or other binding molecule.

In one aspect, the present disclosure provides a method comprising administering to a subject an anti-C1s antibody (e.g., the anti-C1s antibody of Table 1 or Table 2); measuring a level of one or more biomarkers of inflammation (e.g., CRP), or one or more inflammatory cytokines (e.g., IL-6, and/or IL-10) in a sample (e.g., blood, e.g., serum) from the subject. In some embodiments, the subject has a complement-mediated disease, such as CAD. In some embodiments, the subject has fatigue.

In another aspect, the present disclosure provides a method comprising measuring a level of one or more biomarkers of inflammation (e.g., CRP), or one or more inflammatory cytokines (e.g., IL-6, and/or IL-10) in a sample from a subject being treated with an anti-C1s antibody (e.g., the anti-C1s antibody of Table 1 or Table 2). The subject, in some embodiments, has a complement-mediated disease (e.g., CAD). In some embodiments, the subject has fatigue.

Yet other aspects of the present disclosure provide a method comprising treating a subject with an anti-C1s antibody (e.g., the anti-C1s antibody of Table 1 or Table 2), wherein the subject has fatigue, and measuring a level of one or more biomarkers of inflammation (e.g., CRP), or one or more inflammatory cytokines (e.g., IL-6, and/or IL-10) in a sample from the subject. In some embodiments, the subject has a complement-mediated disease, such as CAD.

Further aspects of the present disclosure provide a method comprising treating a subject with an anti-C1s antibody (e.g., the anti-C1s antibody of Table 1 or Table 2), wherein the subject has a complement mediated disease (e.g., CAD), and measuring a level of one or more biomarkers of inflammation (e.g., CRP), or one or more inflammatory cytokines (e.g., IL-6, and/or IL-10) in a sample from the subject. The subject, in some embodiments, has fatigue.

In some embodiments, a level of CRP is measured. CRP, in some embodiments, is used as a surrogate for IL-6. In some embodiments, a level of IL-6 is measured. In some embodiments, a level of IL-10 is measured.

In some embodiments, a method of the present disclosure further comprises assessing fatigue in the subject. In some embodiments, the fatigue is assessed based on a FACIT-F score. The FACIT-F is a 13-item patient-reported outcome instrument that was designed to assess fatigue-related symptoms and impacts on daily functioning (Cella et al. Cancer 94(2):528-238 (2002)); Yellen et al., J Pain Symptom Manage 13(2):63-74 (1997); Lai et al., J Rheumatol 38(4):672-9 (2011); Reddy et al., J Palliat Med 19(5):1068-75 (2007)). Other methods of assessing fatigue may be used. A number of fatigue-measurement scales are known in the art (e.g., Fatigue Severity Scale (FSS), Fatigue Impact Scale (FIS), Brief Fatigue Inventory (BFI), Fatigue Symptom Inventory (FSI), Multidimensional Assessment of Fatigue (MAF), and Multidimensional Fatigue Symptom Inventory (MFSI), etc. (Whitehead, J Pain Symptom Manage 37(1):10-7-28 (2009)). Fatigue may be assessed as part of a multi-symptom scale or a fatigue specific scale (Hjollund et al., Health Qual Life Outcomes 5:12 (2007)).

In some embodiments, the subject has a baseline level of CRP, IL-6, and/or IL-10 prior to treatment with an anti-C1s antibody. In some embodiments, the subject has a baseline level of CRP of greater than 3 mg/mL (e.g., greater than 4 mg/mL, greater than 5 mg/mL, greater than 6 mg/mL, greater than 7 mg/mL, greater than 8 mg/mL, greater than 9 mg/mL, or greater than 10 mg/mL, greater than 20 mg/mL, greater than 50 mg/mL, etc.). In some embodiments, the subject has a baseline level of IL-6 of greater than 1.8 pg/mL (e.g., greater than 2 pg/mL, greater than 2.5 pg/mL, greater than 3 pg/mL, between about 1.8 pg/mL and about 2 pg/mL, between about 2 pg/mL to about 2.5 pg/mL, between about 2.5 to about 3 pg/mL, between about 3 pg/mL to about 3.5 pg/mL, between about 3.5 pg/mL to about 4 pg/mL, etc.). In some embodiments, the subject has a baseline level of IL-10 of greater than 1 pg/mL (e.g., greater than 1.1 pg/mL, greater than 1.2 pg/mL, greater than 1.3 pg/mL, greater than 1.4 pg/mL, greater than 1.5 pg/mL, 2 pg/mL, greater than 2.5 pg/mL, greater than 3 pg/mL, between about 1 pg/mL to about 1.5 pg/mL, between about 1 pg/mL and about 2 pg/mL, between about 2 pg/mL to about 2.5 pg/mL, between about 2.5 to about 3 pg/mL, between about 3 pg/mL to about 3.5 pg/mL, between about 3.5 pg/mL to about 4 pg/mL, etc.). In some embodiments, the subject has a baseline level of fatigue prior to treatment with an anti-C1s antibody. In some embodiments, the baseline level of fatigue is assessed based on a FACIT-F score. In some embodiments, the baseline FACIT-F score of a subject ranges from 20-25, 25-30, 30-35, or 35-40. In some embodiments, the baseline FACIT-F score of a subject is 25, 26, 27, 28, 29, 30, 31, 32, 33, 34, 35, 36, 37, 38, 39, or 40.

It is demonstrated herein that a reduction in the levels of inflammatory cytokines correlates with an improvement in fatigue in subjects who are receiving treatment with an anti-C1s antibody. Accordingly, the levels of the inflammatory cytokines tested have been shown to be indicative of the efficacy of the treatment, for example, to improve fatigue.

In some embodiments, the levels of one or more biomarkers of inflammation (e.g., CRP), or one or more inflammatory cytokines (e.g., IL-6, and/or IL-10) are monitored over a period of time (e.g., 1 week, one month, 6-8 weeks, three months, six months, a year, two years, etc.). In some embodiments, the levels are assessed once a week, biweekly, bimonthly, or monthly over the course of several months (e.g., at least 3, 4, 5, 6, 7, 8, 9, 10, 11, 12, etc.) or over the course of several years (e.g., at least 2, 3, 4, 5, etc.).

In some embodiments, if the level of one or more biomarkers of inflammation (e.g., CRP), or one or more inflammatory cytokines (e.g., IL-6, and/or IL-10) in the sample is reduced, for example, by at least 5%, or by at least 10% (e.g., at least 10%, at least 15%, at least 20%, at least 25%, at least 30%, at least 40%, at least 50%, at least 60%, at least 70%, or at least 80%, between about 5% to about 10%, between about 10% to about 15%, between about 15% to about 20%, between about 20% to about 25%, between about 25% to about 30%, between about 30% to about 40%, between about 40% to about 50%, between about 50% to about 60%, between about 60% to about 80%, etc., or if the levels are reduced to a level that is within the normal range for that biomarker or inflammatory cytokine) relative to baseline and/or fatigue in the subject is improved relative to baseline, the method further comprises continuing with a current anti-C1s antibody treatment. In some embodiments, an improvement in fatigue is an improvement of at least at 3 points (e.g., at least 4 points, at least 5 points, at least 6 points, at least 7 points, at least 8 points, at least 9 points, at least 10 points, at least 11 points, at least 12 points, at least 13 points, at least 14 points, at least 15 points, 3-15 points, 3-10 points, 3-5 points, 5-15 points, 5-10 points, etc.) on the FACIT-F score relative to baseline. In some embodiments, if the level of one or more biomarkers of inflammation (e.g., CRP), or one or more inflammatory cytokines (e.g., IL-6, and/or IL-10) in the sample is within 5% or 10% of baseline and/or fatigue the subject is maintained or worsens relative to baseline, the method further comprises altering a current anti-C1s antibody treatment. In some embodiments, the fatigue is assessed based on a FACIT-F score. In some embodiments, a worsening in fatigue is a decrease of at least at 3 points (e.g., at least 4 points, at least 5 points, at least 6 points, at least 7 points, at least 8 points, at least 9 points, at least 10 points, at least 11 points, at least 12 points, at least 13 points, at least 14 points, at least 15 points, 3-15 points, 3-10 points, 3-5 points, 5-15 points, 5-10 points, etc.) on the FACIT-F score relative to baseline.

In some embodiments, the levels of one or more biomarkers of inflammation (e.g., CRP), or one or more inflammatory cytokines (e.g., IL-6, and/or IL-10) are measured at a first point in time and a second point in time after commencing treatment with the anti-C1s antibody. In some embodiments, the first point in time and the second point in time are a few days apart (e.g., less than 1 week), 1 week apart, 2 weeks apart, 3 weeks apart, 1 month apart, 2 months apart, or several months apart (e.g., at least 3, 4, 5, 6, 7, 8, 9, 10, 11, 12, etc.). In some embodiments, if the level of one or more biomarkers of inflammation (e.g., CRP), or one or more inflammatory cytokines (e.g., IL-6, and/or IL-10) in the sample is reduced, for example, by at least 5% or by at least 10% (e.g., at least 5%, at least 10%, at least 15%, at least 20%, at least 25%, at least 30%, at least 40%, at least 50%, at least 60%, at least 70%, or at least 80%, between about 5% to about 10%, between about 10% to about 15%, between about 15% to about 20%, between about 20% to about 25%, between about 25% to about 30%, between about 30% to about 40%, between about 40% to about 50%, between about 50% to about 60%, between about 60% to about 80%, etc., or if the levels are reduced to a level that is within the normal range for that biomarker or inflammatory cytokine) at the second time point relative to the first time point and/or fatigue in the subject is improved relative to the first time point, the method further comprises continuing with a current anti-C1s antibody treatment. In some embodiments, if the level of one or more biomarkers of inflammation (e.g., CRP), or one or more inflammatory cytokines (e.g., IL-6, and/or IL-10) in the sample at the second time point is within 5% or 10% of the level at the first time point and/or fatigue the subject is maintained or worsens relative to the first time point, the method further comprises altering a current anti-C1s antibody treatment. In some embodiments, a worsening in fatigue is a decrease of at least at 3 points (e.g., at least 4 points, at least 5 points, at least 6 points, at least 7 points, at least 8 points, at least 9 points, at least 10 points, at least 11 points, at least 12 points, at least 13 points, at least 14 points, at least 15 points, 3-15 points, 3-10 points, 3-5 points, 5-15 points, 5-10 points, etc.) on the FACIT-F score relative to baseline.

In some embodiments, where a patient weighs <75 kg, continuing with a current anti-C1s antibody treatment comprises: a) administering an effective dose of about 6.5 g of the anti-C1s antibody on Day 1; b) administering an effective dose of about 6.5 g of the anti-C1s antibody on Day 8; and c) administering an effective dose of about 6.5 g of the anti-C1s antibody every other week following the Day 8 administration. In some embodiments, an effective dose of about 6.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some embodiments, an effective dose of about 6.5 g of the anti-C1s antibody is administered to the individual every other week for a period of time of more than 1 year. For example, in some embodiments, an effective dose of about 6.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years. In some embodiments, where a patient weighs ≥75 kg, continuing with a current anti-C1s antibody treatment comprises: a) administering an effective dose of about 7.5 g of the anti-C1s antibody on Day 1; b) administering an effective dose of about 7.5 g of the anti-C1s antibody on Day 8; and c) administering an effective dose of about 7.5 g of the anti-C1s antibody every other week following the Day 8 administration. In some embodiments, an effective dose of about 7.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some embodiments, an effective dose of about 7.5 g of the anti-C1s antibody is administered to the individual every other week for a period of time of more than 1 year. For example, in some embodiments, an effective dose of about 7.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In some embodiments, altering treatment with the anti-C1s antibody comprises adjusting the dosage and/or frequency of the treatment with the anti-C1s antibody. In some embodiments, adjusting the dosage and/or frequency of the treatment with the anti-C1s antibody involves increasing the dosage and/or frequency of the treatment with the anti-C1s antibody. In some embodiments, the effective dose of the anti-C1s antibody is increased by about 0.1 g to about 0.5 g, about 0.5 g to about 1 g, about 1 g to about 1.5 g, about 1.5 g to about 2.0 g, about 2.0 to about 2.5 g, about 2.5 g to about 3 g, or about 3 g to about 3.5 g. In some embodiments, the effective dose of the anti-C1s antibody is increased by about 0.1 g, about 0.2 g, about 0.3 g, about 0.4 g, about 0.5 g, about 0.6 g, about 0.7 g, about 0.8 g, about 0.9 g, about 1 g, about 1.5 g, about 2 g, about 2.5 g, about 3 g, or about 3.5 g. In some embodiments, the frequency of the treatment is increased to monthly, bi-weekly, weekly, every other day, or daily. In some embodiments, the anti-C1s antibody is administered as one or more loading doses followed by dosing at dosing intervals. In some embodiments, the frequency of the loading doses is increased to monthly, bi-weekly, weekly, every other day, or daily. In some embodiments, the dosing interval following the initial one or more loading doses is reduced (e.g., reduced to monthly, bi-weekly, weekly, every other day, daily, etc.). In some embodiments, the number of loading doses is increased (e.g., by 1, 2, 3, 4, or more).

In some embodiments, altering treatment with the anti-C1s antibody comprises further treatment of the subject with an anti-inflammatory agent. In some embodiments, an anti-inflammatory agent is selected from one or more of: a glucocorticoid (e.g., cortisol, prednisolone, methyl-prednisolone, dexamethasone); a nonsteroidal anti-inflammatory drug (NSAID) (e.g., aspirin, ibuprofen, fenoprofen, naproxen, sulindac, diclofenac, piroxicam, ketoprofen, diflunisal, nabumetone, etodolac, or oxaprozin, indomethacin); Cox-2 inhibitors (e.g., rofecoxib and celecoxib); interferon, interferon derivatives including betaseron, beta-interferon; soluble TNF-receptors; anti-TNF-antibodies; soluble receptors of interleukins or other cytokines (e.g., receptors of IL-6, IL-8, IL-10, IL-12, IL-17, IL-18, IL-1β, or IFN-γ); antibodies against interleukins or other cytokines (e.g., IL-6, IL-8, IL-10, IL-12, IL-17, IL-18, IL-1β, or IFN-γ); and antibodies against receptors of interleukins or other cytokines (e.g., receptors of IL-6, IL-8, IL-10, IL-12, IL-17, IL-18, IL-1β, or IFN-γ). In some embodiments, the anti-inflammatory agent is an IL-6 antagonist (e.g., an anti-IL-6 antibody). In some embodiments, the anti-inflammatory agent is an IL-10 antagonist (e.g., an anti-IL-10 antibody).

In some embodiments, altering treatment with the anti-C1s antibody comprises further treatment of the subject to improve fatigue. In some embodiments, the further treatment to improve fatigue comprises administering one or more of: an NSAID (e.g., aspirin, ibuprofen, fenoprofen, naproxen, sulindac, diclofenac, piroxicam, ketoprofen, diflunisal, nabumetone, etodolac, oxaprozin, or indomethacin); an antihistamine (diphenhydramine or doxylamine); a stimulant (e.g., modafinil, armodafinil, methylphenidate, dexamphetamine, or amphetamine salts); a sleep aid (e.g., melatonin, cyclobenzaprine, clonazepam, zolpidem, zopiclone, or promethazine), an antiseizure drug (e.g., gabapentin, or pregabalin); an antidepressant (e.g., amitriptyline, doxepin, nortriptyline, trazodone, or mirtazapine); a pain reliever (e.g., acetaminophen, oxycodone, hydrocodone, morphine, fentanyl, buprenorphine, tapendatol, or tramadol).

In some embodiments, the subject has undergone a blood transfusion. In some embodiments, the subject has undergone a blood transfusion prior to commencing treatment with an anti-C1s antibody, e.g., within a month, within 3 weeks, within 2 weeks, or within 1 week.

### Humanized Anti-C1s Antibodies

An exemplary humanized anti-C1s antibody sequence is provided in Table 1 below.

TABLE 1
Anti-C1s Antibody #1 (Sutimlimab)

| Anti- | | SEQ |
|---|---|---|
| body | | ID |
| Region | Sequence | NO: |
| HC | EVQLVESGGGLVKPGGSLRLSCAASGFTFSNYAMSWVR | 1 |
| | QAPGKGLEWVATISSGGSHTYYLDSVKGRFTISRDNSK | |
| | NTLYLQMNSLRAEDTALYYCARLFTGYAMDYWGQGTLV | |
| | TVSSASTKGPSVFPLAPCSRSTSESTAALGCLVKDYFP | |
| | EPVTVSWNSGALTSGVHTFPAVLQSSGLYSLSSVVTVP | |
| | SSSLGTKTYTCNVDHKPSNTKVDKRVESKYGPPCPPCP | |
| | APEFEGGPSVFLFPPKPKDTLMISRTPEVTCVVVDVSQ | |
| | EDPEVQFNWYVDGVEVHNAKTKPREEQFNSTYRVVSVL | |
| | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGK | |
| LC | QIVLTQSPATLSLSPGERATMSCTASSSVSSSYLHWYQ | 2 |
| | QKPGKAPKLWIYSTSNLASGVPSRFSGSGSGTDYTLTI | |
| | SSLQPEDFATYYCHQYYRLPPITFGQGTKLEIKRTVAA | |
| | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| VH | EVQLVESGGGLVKPGGSLRLSCAASGFTFSNYAMSWVR | 3 |
| | QAPGKGLEWVATISSGGSHTYYLDSVKGRFTISRDNSK | |
| | NTLYLQMNSLRAEDTALYYCARLFTGYAMDYWGQGTLV | |
| | TVSS | |
| VL | QIVLTQSPATLSLSPGERATMSCTASSSVSSSYLHWYQ | 4 |
| | QKPGKAPKLWIYSTSNLASGVPSRFSGSGSGTDYTLTI | |
| | SSLQPEDFATYYCHQYYRLPPITFGQGTKLEIK | |
| HC | NYAMS | 5 |
| CDR1 | | |
| HC | TISSGGSHTYYLDSVKG | 6 |
| CDR2 | | |
| HC | LFTGYAMDY | 7 |
| CDR3 | | |
| LC | TASSSVSSSYLH | 8 |
| CDR1 | | |
| LC | STSNLAS | 9 |
| CDR2 | | |
| LC | HQYYRLPPIT | 10 |
| CDR3 | | |

Residue numbering follows the nomenclature of Kabat et al., U.S. Dept. of Health and Human Services, “Sequences of proteins of immunological interest” (1991).

In some embodiments, a humanized anti-C1s antibody comprises a heavy chain complementarity determining region 1 (HC CDR1) comprising the amino acid sequence of SEQ ID NO: 5. In some embodiments, a humanized anti-C1s antibody comprises a heavy chain complementarity determining region 2 (HC CDR2) comprising the amino acid sequence of SEQ ID NO: 6. In some embodiments, a humanized anti-C1s antibody comprises a heavy chain complementarity determining region 3 (HC CDR3) comprising the amino acid sequence of SEQ ID NO: 7. In some embodiments, a humanized anti-C1s antibody comprises an HC CDR1 comprising the amino acid sequence of SEQ ID NO: 5, an HC CDR2 comprising the amino acid sequence of SEQ ID NO: 6, and an HC CDR3 comprising the amino acid sequence of SEQ ID NO: 7.

In some embodiments, a humanized anti-C1s antibody comprises a light chain complementarity determining region 1 (LC CDR1) comprising the amino acid sequence of SEQ ID NO: 8. In some embodiments, a humanized anti-C1s antibody comprises a light chain complementarity determining region 2 (LC CDR2) comprising the amino acid sequence of SEQ ID NO: 9. In some embodiments, a humanized anti-C1s antibody comprises a light chain complementarity determining region 3 (LC CDR3) comprising the amino acid sequence of SEQ ID NO: 10. In some embodiments, a humanized anti-C1s antibody comprises an LC CDR1 comprising the amino acid sequence of SEQ ID NO: 8, an LC CDR2 comprising the amino acid sequence of SEQ ID NO: 9, and an LC CDR3 comprising the amino acid sequence of SEQ ID NO: 10.

In some embodiments, a humanized anti-C1s antibody comprises an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, an LC CDR1 comprising the amino acid sequence of SEQ ID NO: 8, an LC CDR2 comprising the amino acid sequence of SEQ ID NO: 9, and an LC CDR3 comprising the amino acid sequence of SEQ ID NO: 10.

In some embodiments, a humanized anti-C1s antibody comprises a heavy chain variable region (VH) comprising the amino acid sequence of SEQ ID NO: 3.

In some embodiments, a humanized anti-C1s antibody comprises a light chain variable region (VL) comprising the amino acid sequence of SEQ ID NO: 4.

In some embodiments, a humanized anti-C1s antibody comprises a VH comprising the amino acid sequence of SEQ ID NO: 3 and a VL comprising the amino acid sequence of SEQ ID NO: 4.

In some embodiments, a humanized anti-C1s antibody comprises a heavy chain (HC) comprising the amino acid sequence of SEQ ID NO: 1.

In some embodiments, a humanized anti-C1s antibody comprises a light chain (LC) comprising the amino acid sequence of SEQ ID NO: 2.

In some embodiments, a humanized anti-C1s antibody comprises a HC comprising the amino acid sequence of SEQ ID NO: 1 and a LC comprising the amino acid sequence of SEQ ID NO: 2.

In some embodiments, a humanized anti-C1s antibody comprises an HC CDR1 comprising an amino acid sequence containing no more than 3 amino acid variations (e.g., no more than 3, 2, or 1 amino acid variation(s)) relative to the HC CDR1 amino acid sequence of SEQ ID NO: 5. In some embodiments, a humanized anti-C1s antibody comprises an HC CDR2 comprising an amino acid sequence containing no more than 3 amino acid variations (e.g., no more than 3, 2, or 1 amino acid variation(s)) relative to the HC CDR2 amino acid sequence of SEQ ID NO: 6. In some embodiments, a humanized anti-C1s antibody comprises an HC CDR3 comprising an amino acid sequence containing no more than 3 amino acid variations (e.g., no more than 3, 2, or 1 amino acid variation(s)) relative to the HC CDR3 amino acid sequence of SEQ ID NO: 7. In some embodiments, affinity maturation may be used to identify CDR variations that preserve binding specificity.

In some embodiments, a humanized anti-C1s antibody comprises an LC CDR1 comprising an amino acid sequence containing no more than 3 amino acid variations (e.g., no more than 3, 2, or 1 amino acid variation(s)) relative to the LC CDR1 amino acid sequence of SEQ ID NO: 8. In some embodiments, a humanized anti-C1s antibody comprises an LC CDR2 comprising an amino acid sequence containing no more than 3 amino acid variations (e.g., no more than 3, 2, or 1 amino acid variation(s)) relative to the LC CDR2 amino acid sequence of SEQ ID NO: 9. In some embodiments, a humanized anti-C1s antibody comprises an LC CDR3 comprising an amino acid sequence containing no more than 3 amino acid variations (e.g., no more than 3, 2, or 1 amino acid variation(s)) relative to the LC CDR3 amino acid sequence of SEQ ID NO: 10.

In some embodiments, a humanized anti-C1s antibody comprises a VH comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH amino acid sequence of SEQ ID NO: 3.

In some embodiments, a humanized anti-C1s antibody comprises a VL comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL amino acid sequence of SEQ ID NO: 4.

In some embodiments, a humanized anti-C1s antibody comprises a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 3.

In some embodiments, a humanized anti-C1s antibody comprises a VL comprising an LC CDR1 comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 4.

In some embodiments, a humanized anti-C1s antibody comprises (a) a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 3, and (b) a VL comprising an LC CDR1 comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 4.

In some embodiments, a humanized anti-C1s antibody comprises a VH comprising an amino acid sequence having at least 80% (e.g., 80%, 85%, 90%, 95%, 96%, 97%, 98%, or 99%) identity to the VH amino acid sequence of SEQ ID NO: 3.

In some embodiments, a humanized anti-C1s antibody comprises a VL comprising an amino acid sequence having at least 80% (e.g., 80%, 85%, 90%, 95%, 96%, 97%, 98%, or 99%) identity to the VL amino acid sequence of SEQ ID NO: 4.

In some embodiments, a humanized anti-C1s antibody comprises a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, and comprises framework regions that have at least 80% (e.g., 80%, 85%, 90%, 95%, 96%, 97%, 98%, or 99%) identity to the framework regions of the VH sequence of SEQ ID NO: 3.

In some embodiments, a humanized anti-C1s antibody comprises a VL comprising an LC CDR1 comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that have at least 80% (e.g., 80%, 85%, 90%, 95%, 96%, 97%, 98%, or 99%) identity to the framework regions of the VL sequence of SEQ ID NO: 4.

In some embodiments, a humanized anti-C1s antibody comprises (a) a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, and comprises framework regions that have at least 80% (e.g., 80%, 85%, 90%, 95%, 96%, 97%, 98%, or 99%) identity to the framework regions of the VH sequence of SEQ ID NO: 3, and (b) a VL comprising an LC CDR1 comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that have at least 80% (e.g., 80%, 85%, 90%, 95%, 96%, 97%, 98%, or 99%) identity to the framework regions of the VL sequence of SEQ ID NO: 4.

Another exemplary humanized anti-C1s antibody sequence is provided in Table 2 below.

TABLE 2
Anti-C1s Antibody #2

| Anti- | | SEQ |
|---|---|---|
| body | | ID |
| Region | Sequence | NO: |
| HC | QVQLVQSGAEVKKPGASVKLSCTASGFNIKDDYIHWVK | 11 |
| | QAPGQGLEWIGRIDPADGHTKYAPKFQVKVTITADTST | |
| | STAYLELSSLRSEDTAVYYCARYGYGREVFDYWGQGTT | |
| | VTVSSASTKGPSVFPLAPCSRSTSESTAALGCLVKDYF | |
| | PEPVTVSWNSGALTSGVHTFPAVLQSSGLYSLSSVVTV | |
| | PSSSLGTKTYTCNVDHKPSNTKVDKRVESKYGPPCPPC | |
| | PAPEFEGGPSVFLFPPKPKDTLMISRTPEVTCVVVDVS | |
| | QEDPEVQFNWYVDGVEVHNAKTKPREEQFNSTYRVVSV | |
| | LTVLHQDWLNGKEYKCKVSNKGLPSSIEKTISKAKGQP | |
| | REPQVYTLPPSQEEMTKNQVSLTCLVKGFYPSDIAVEW | |
| | ESNGQPENNYKTTPPVLDSDGSFFLYSRLTVDKSRWQE | |
| | GNVFSCSVLHEALHSHYTQKSLSLSLGK | |
| LC | DIVLTQSPDSLAVSLGERATISCKASQSVDYDGDSYMN | 12 |
| | WYQQKPGQPPKILIYDASNLESGIPARFSGSGSGTDFT | |
| | LTISSLEPEDFAIYYCQQSNEDPWTFGGGTKVEIKRTV | |
| | AAPSVFIFPPSDEQLKSGTASVVCLLNNFYPREAKVQW | |
| | VDNALQSGNSQESVTEQDSKDSTYSLSSTLTLSKAD | |
| | KYEKHKVYACEVTHQGLSSPVTKSFNRGEC | |
| VH | QVQLVQSGAEVKKPGASVKLSCTASGFNIKDDYIHWVK | 13 |
| | QAPGQGLEWIGRIDPADGHTKYAPKFQVKVTITADTST | |
| | STAYLELSSLRSEDTAVYYCARYGYGREVFDYWGQGTT | |
| | VTVSS | |
| VL | DIVLTQSPDSLAVSLGERATISCKASQSVDYDGDSYMN | 14 |
| | WYQQKPGQPPKILIYDASNLESGIPARFSGSGSGTDFT | |
| | LTISSLEPEDFAIYYCQQSNEDPWTFGGGTKVEIK | |
| HC | DDYIH | 15 |
| CDR1 | | |
| HC | RIDPADGHTKYAPKFQV | 16 |
| CDR2 | | |
| HC | YGYGREVFDY | 17 |
| CDR3 | | |
| LC | KASQSVDYDGDSYMN | 18 |
| CDR1 | | |
| LC | DASNLES | 19 |
| CDR2 | | |
| LC | QQSNEDPWT | 20 |
| CDR3 | | |

Residue numbering follows the nomenclature of Kabat et al., U.S. Dept. of Health and Human Services, “Sequences of proteins of immunological interest” (1991).

In some embodiments, a humanized anti-C1s antibody comprises a heavy chain complementarity determining region 1 (HC CDR1) comprising the amino acid sequence of SEQ ID NO: 15. In some embodiments, a humanized anti-C1s antibody comprises a heavy chain complementarity determining region 2 (HC CDR2) comprising the amino acid sequence of SEQ ID NO: 16. In some embodiments, a humanized anti-C1s antibody comprises a heavy chain complementarity determining region 3 (HC CDR3) comprising the amino acid sequence of SEQ ID NO: 17. In some embodiments, a humanized anti-C1s antibody comprises an HC CDR1 comprising the amino acid sequence of SEQ ID NO: 15, an HC CDR2 comprising the amino acid sequence of SEQ ID NO: 16, and an HC CDR3 comprising the amino acid sequence of SEQ ID NO: 17.

In some embodiments, a humanized anti-C1s antibody comprises a light chain complementarity determining region 1 (LC CDR1) comprising the amino acid sequence of SEQ ID NO: 18. In some embodiments, a humanized anti-C1s antibody comprises a light chain complementarity determining region 2 (LC CDR2) comprising the amino acid sequence of SEQ ID NO: 19. In some embodiments, a humanized anti-C1s antibody comprises a light chain complementarity determining region 3 (LC CDR3) comprising the amino acid sequence of SEQ ID NO: 20. In some embodiments, a humanized anti-C1s antibody comprises an LC CDR1 comprising the amino acid sequence of SEQ ID NO: 18, an LC CDR2 comprising the amino acid sequence of SEQ ID NO: 19, and an LC CDR3 comprising the amino acid sequence of SEQ ID NO: 20.

In some embodiments, a humanized anti-C1s antibody comprises an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, an LC CDR1 comprising the amino acid sequence of SEQ ID NO: 18, an LC CDR2 comprising the amino acid sequence of SEQ ID NO: 19, and an LC CDR3 comprising the amino acid sequence of SEQ ID NO: 20.

In some embodiments, a humanized anti-C1s antibody comprises a heavy chain variable region (VH) comprising the amino acid sequence of SEQ ID NO: 13.

In some embodiments, a humanized anti-C1s antibody comprises a light chain variable region (VL) comprising the amino acid sequence of SEQ ID NO: 14.

In some embodiments, a humanized anti-C1s antibody comprises a VH comprising the amino acid sequence of SEQ ID NO: 13 and a VL comprising the amino acid sequence of SEQ ID NO: 14.

In some embodiments, a humanized anti-C1s antibody comprises a heavy chain (HC) comprising the amino acid sequence of SEQ ID NO: 11.

In some embodiments, a humanized anti-C1s antibody comprises a light chain (LC) comprising the amino acid sequence of SEQ ID NO: 12.

In some embodiments, a humanized anti-C1s antibody comprises a HC comprising the amino acid sequence of SEQ ID NO: 11 and a LC comprising the amino acid sequence of SEQ ID NO: 12.

In some embodiments, a humanized anti-C1s antibody comprises an HC CDR1 comprising an amino acid sequence containing no more than 3 amino acid variations (e.g., no more than 3, 2, or 1 amino acid variation(s)) relative to the HC CDR1 amino acid sequence of SEQ ID NO: 15. In some embodiments, a humanized anti-C1s antibody comprises an HC CDR2 comprising an amino acid sequence containing no more than 3 amino acid variations (e.g., no more than 3, 2, or 1 amino acid variation(s)) relative to the HC CDR2 amino acid sequence of SEQ ID NO: 16. In some embodiments, a humanized anti-C1s antibody comprises an HC CDR3 comprising an amino acid sequence containing no more than 3 amino acid variations (e.g., no more than 3, 2, or 1 amino acid variation(s)) relative to the HC CDR3 amino acid sequence of SEQ ID NO: 7.

In some embodiments, a humanized anti-C1s antibody comprises an LC CDR1 comprising an amino acid sequence containing no more than 3 amino acid variations (e.g., no more than 3, 2, or 1 amino acid variation(s)) relative to the LC CDR1 amino acid sequence of SEQ ID NO: 18. In some embodiments, a humanized anti-C1s antibody comprises an LC CDR2 comprising an amino acid sequence containing no more than 3 amino acid variations (e.g., no more than 3, 2, or 1 amino acid variation(s)) relative to the LC CDR2 amino acid sequence of SEQ ID NO: 19. In some embodiments, a humanized anti-C1s antibody comprises an LC CDR3 comprising an amino acid sequence containing no more than 3 amino acid variations (e.g., no more than 3, 2, or 1 amino acid variation(s)) relative to the LC CDR3 amino acid sequence of SEQ ID NO: 20.

In some embodiments, a humanized anti-C1s antibody comprises a VH comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH amino acid sequence of SEQ ID NO: 13.

In some embodiments, a humanized anti-C1s antibody comprises a VL comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL amino acid sequence of SEQ ID NO: 14.

In some embodiments, a humanized anti-C1s antibody comprises a VH comprising an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 13.

In some embodiments, a humanized anti-C1s antibody comprises a VL comprising an LC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 14.

In some embodiments, a humanized anti-C1s antibody comprises (a) a VH comprising an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 13, and (b) a VL comprising an LC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 14.

In some embodiments, a humanized anti-C1s antibody comprises a VH comprising an amino acid sequence having at least 80% (e.g., 80%, 85%, 90%, 95%, 96%, 97%, 98%, or 99%) identity to the VH amino acid sequence of SEQ ID NO: 13.

In some embodiments, a humanized anti-C1s antibody comprises a VL comprising an amino acid sequence having at least 80% (e.g., 80%, 85%, 90%, 95%, 96%, 97%, 98%, or 99%) identity to the VL amino acid sequence of SEQ ID NO: 14.

In some embodiments, a humanized anti-C1s antibody comprises a VH comprising an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, and comprises framework regions that have at least 80% (e.g., 80%, 85%, 90%, 95%, 96%, 97%, 98%, or 99%) identity to the framework regions of the VH sequence of SEQ ID NO: 13.

In some embodiments, a humanized anti-C1s antibody comprises a VL comprising an LC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20, and comprises framework regions that have at least 80% (e.g., 80%, 85%, 90%, 95%, 96%, 97%, 98%, or 99%) identity to the framework regions of the VL sequence of SEQ ID NO: 14.

In some embodiments, a humanized anti-C1s antibody comprises (a) a VH comprising an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, and comprises framework regions that have at least 80% (e.g., 80%, 85%, 90%, 95%, 96%, 97%, 98%, or 99%) identity to the framework regions of the VH sequence of SEQ ID NO: 13, and (b) a VL comprising an LC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20, and comprises framework regions that have at least 80% (e.g., 80%, 85%, 90%, 95%, 96%, 97%, 98%, or 99%) identity to the framework regions of the VL sequence of SEQ ID NO: 14.

“Antibody” encompasses antibodies or immunoglobulins of any isotype, including but not limited to humanized antibodies and chimeric antibodies. An antibody may be a single-chain antibody (scAb) or a single domain antibody (dAb) (e.g., a single domain heavy chain antibody or a single domain light chain antibody; see Holt et al. (2003) Trends Biotechnol. 21:484). The term “antibody” also encompasses fragments of antibodies (antibody fragments) that retain specific binding to an antigen. “Antibody” further includes single-chain variable fragments (scFvs), which are fusion proteins of the variable regions of the heavy (V_{H}) and light chains (V_{L}) of antibodies, connected with a short linker peptide, and diabodies, which are noncovalent dimers of scFv fragments that include the VH and VL connected by a small peptide linker (Zapata et al., Protein Eng. 8(10): 1057-1062 (1995)). Other fusion proteins that comprise an antigen-binding portion of an antibody and a non-antibody protein are also encompassed by the term “antibody.”

“Antibody fragments” comprise a portion of an intact antibody, for example, the antigen binding or variable region of the intact antibody. Examples of antibody fragments include an antigen-binding fragment (Fab), Fab′, F(ab′)₂, a variable domain Fv fragment (Fv), an Fd fragment, and an antigen binding fragment of a chimeric antigen receptor.

Papain digestion of antibodies produces two identical antigen-binding fragments, referred to as “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region includes a dimer of one heavy-chain variable domain and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” fragments contain the constant domain of the light chain and the first constant domain (CH₁) of the heavy chain. Fab fragments differ from Fab′ fragments by the addition of a few residues at the carboxyl terminus of the heavy chain CH₁ domain including at least one cysteine from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“scFv” antibody fragments comprise the V_{H} and V_{L} of an antibody, wherein these regions are present in a single polypeptide chain. In some embodiments, the Fv polypeptide further comprises a polypeptide linker between the V_{H} and V_{L} regions, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

“Diabody” refers to a small antibody fragment with two antigen-binding sites, which fragments comprise a V_{H} connected to a V_{L} in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, Hollinger et al. Proc. Natl. Acad. Sci. USA 90: 6444-6448 (1993).

An antibody can be monovalent or bivalent. An antibody can be an Ig monomer, which is a “Y-shaped” molecule that consists of four polypeptide chains: two heavy chains and two light chains connected by disulfide bonds.

Antibodies can be detectably labeled, e.g., with a radioisotope, an enzyme that generates a detectable product, and/or a fluorescent protein. Antibodies can be further conjugated to other moieties, such as members of specific binding pairs, e.g., biotin member of biotin-avidin specific binding pair. Antibodies can also be bound to a solid support, including, but not limited to, polystyrene plates and/or beads.

An “isolated” antibody is one that has been identified and separated and/or recovered from a component of its natural environment (i.e., is not naturally occurring). Contaminant components of its natural environment are materials that would interfere with uses (e.g., diagnostic or therapeutic uses) of the antibody, and can include enzymes, hormones, and other proteinaceous or nonproteinaceous solutes. In some embodiments, an antibody is purified (1) to greater than 90%, greater than 95%, or greater than 98% by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or non-reducing conditions using Coomassie blue or silver stain. Isolated antibodies encompass antibodies in situ within recombinant cells, as at least one component of the antibody's natural environment will not be present. In some embodiments, an isolated antibody is prepared by at least one purification step.

A “monoclonal antibody” is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries).

A “complementarity determining region (CDR)” is the non-contiguous antigen combining sites found within the variable region of both heavy and light chain polypeptides. CDRs have been described by Lefranc et al. (2003) Developmental and Comparative Immunology 27:55; Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

The terms “LC CDR1,” “LC CDR2,” and “LC CDR3” refer, respectively, to the first, second, and third CDRs in a light chain variable region. As used herein, the terms “HC CDR1”, “HC CDR2”, and “HC CDR3” refer, respectively, to the first, second, and third CDRs in a heavy chain variable region. As used herein, the terms “CDR1”, “CDR2”, and “CDR3” refer, respectively, to the first, second and third CDRs of either chain's variable region.

A “framework” when used in reference to an antibody variable region includes all amino acid residues outside the CDR regions within the variable region of an antibody. A variable region framework is generally a discontinuous amino acid sequence that includes only those amino acids outside of the CDRs. A “framework region” includes each domain of the framework that is separated by the CDRs.

A “humanized antibody” is an antibody comprising portions of antibodies of different origin, wherein at least one portion comprises amino acid sequences of human origin. For example, the humanized antibody can comprise portions derived from an antibody of nonhuman origin with the requisite specificity, such as a mouse, and from antibody sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized antibody is an antibody containing at least one chain comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

In some embodiments, a humanized antibody is produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

In some embodiments, a humanized anti-C1s antibody described herein is a full-length IgG, an Ig monomer, a Fab fragment, a F(ab′)2 fragment, a Fd fragment, a scFv, a scAb, or a Fv. In some embodiments, a humanized anti-C1s antibody described herein is a full-length IgG. In some embodiments, the heavy chain of any of the humanized anti-C1s antibodies as described herein comprises a heavy chain constant region (CH) or a portion thereof (e.g., CH1, CH2, CH3, or a combination thereof). The heavy chain constant region can of any suitable origin, e.g., human, mouse, rat, or rabbit. In some embodiments, the heavy chain constant region is from a human IgG (a gamma heavy chain), e.g., IgG1, IgG2, or IgG4.

In some embodiments, mutations can be introduced into the heavy chain constant region of any one of the humanized anti-C1s antibodies described herein. In some embodiments, one, two or more mutations (e.g., amino acid substitutions) are introduced into the heavy chain constant region (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more mutations (e.g., amino acid substitutions) are introduced into the hinge region of the heavy chain constant region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof to alter (e.g., decrease or increase) half-life of the antibody in vivo. In some embodiments, the one or more mutations are introduced into an Fc or hinge-Fc domain fragment. See, e.g., International Publication Nos. WO 02/060919; WO 98/23289; and WO 97/34631; and U.S. Pat. Nos. 5,869,046; 6,121,022; 6,277,375; and 6,165,745 for examples of mutations that will alter (e.g., decrease or increase) the half-life of an antibody in vivo.

In some embodiments, the constant region antibody described herein is an IgG1 constant region and comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat. Additional mutations that may be introduced to the heavy chain constant region that would increase the half-life of the antibody are known in the art, e.g., the M428L/N434S (EU numbering; M459L/N466S Kabat numbering) mutations as described in Zalevsky et al., Nat Biotechnol. 2010 February; 28(2): 157-159.

In some embodiments, one, two or more amino acid substitutions are introduced into an IgG constant domain Fc region to alter the effector function(s) of the antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, at least one amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, at least one amino acid in the constant region can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al.). In some embodiments, at least one amino acid residue in the N-terminal region of the CH2 domain of an antibody described herein is altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fey receptor. This approach is described further in International Publication No. WO 00/42072.

In some embodiments, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. In some embodiments, to reduce residual antibody-dependent cellular cytotoxicity, a L235E (EU numbering, corresponding to L248E in Kabat numbering) mutation is introduced to the heavy chain constant region, e.g., as described in Benhnia et al., JOURNAL OF VIROLOGY, December 2009, p. 12355-12367.

In some embodiments, the heavy chain constant region in any one of the humanized anti-C1s antibodies described herein is an IgG4 constant region, or a variant there of. Examples of IgG4 constant regions and variants are provided in Table 3.

TABLE 3
Examples of Heavy Chain Constant Regions

| Heavy Chain | |
|---|---|
| Constant | |
| Region | Amino Acid Sequence |
| IgG4 | ASTKGPSVFPLAPCSRSTSESTAALGCLVKD |
| constant | YFPEPVTVSWNSGALTSGVHTFPAVLQSSGL |
| region WT | YSLSSVVTVPSSSLGTKTYTCNVDHKPSNTK |
| (also re- | VDKRVESKYGPPCPSCPAPEFLGGPSVFLFP |
| ferred to | PKPKDTLMISRTPEVTCVVVDVSQEDPEVQF |
| herein as | NWYVDGVEVHNAKTKPREEQFNSTYRVVSVL |
| “IgG4wt”) | TVLHQDWLNGKEYKCKVSNKGLPSSIEKTIS |
| | KAKGQPREPQVYTLPPSQEEMTKNQVSLTCL |
| | VKGFYPSDIAVEWESNGQPENNYKTTPPVLD |
| | SDGSFFLYSRLTVDKSRWQEGNVFSCSVMHE |
| | ALHNHYTQKSLSLSLGK |
| | (SEQ ID NO: 21) |
| IgG4 | ASTKGPSVFPLAPCSRSTSESTAALGCLVKD |
| constant | YFPEPVTVSWNSGALTSGVHTFPAVLQSSGL |
| region | YSLSSVVTVPSSSLGTKTYTCNVDHKPSNTK |
| variant | VDKRVESKYGPPCPPCPAPEFEGGPSVFLFP |
| 1 (also | PKPKDTLMISRTPEVTCVVVDVSQEDPEVQF |
| referred | NWYVDGVEVHNAKTKPREEQFNSTYRVVSVL |
| to herein | TVLHQDWLNGKEYKCKVSNKGLPSSIEKTIS |
| as | KAKGQPREPQVYTLPPSQEEMTKNQVSLTCL |
| “IgG4v1”) | VKGFYPSDIAVEWESNGQPENNYKTTPPVLD |
| | SDGSFFLYSRLTVDKSRWQEGNVFSCSVMHE |
| | ALHNHYTQKSLSLSLGK |
| | (SEQ ID NO: 22) |
| IgG4 | ASTKGPSVFPLAPCSRSTSESTAALGCLVKD |
| constant | YFPEPVTVSWNSGALTSGVHTFPAVLQSSGL |
| region | YSLSSVVTVPSSSLGTKTYTCNVDHKPSNTK |
| variant | VDKRVESKYGPPCPPCPAPEFEGGPSVFLFP |
| 2 (also | PKPKDTLMISRTPEVTCVVVDVSQEDPEVQF |
| referred | NWYVDGVEVHNAKTKPREEQFNSTYRVVSVL |
| to herein | TVLHQDWLNGKEYKCKVSNKGLPSSIEKTIS |
| as | KAKGQPREPQVYTLPPSQEEMTKNQVSLTCL |
| “IgG4v2”) | VKGFYPSDIAVEWESNGQPENNYKTTPPVLD |
| | SDGSFFLYSRLTVDKSRWQEGNVFSCSVLHE |
| | ALHSHYTQKSLSLSLGK |
| | (SEQ ID NO: 23) |

In some embodiments, the light chain of any of the humanized anti-C1s antibodies described herein may further comprise a light chain constant region (C_{L}). In some examples, the C_{L} is a kappa light chain. In other examples, the C_{L} is a lambda light chain. In some embodiments, the C_{L} is a kappa light chain, the sequence of which is provided below:

(SEQ ID NO: 24)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (www.imgt.org) or at www.vbase2.org/vbstat.php., both of which are incorporated by reference herein.

### Compositions

An anti-C1s antibody is generally present in a composition, e.g., a pharmaceutical composition.

A composition comprising an anti-C1s antibody, in some embodiments, comprises one or more of a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; and/or glycerol.

An anti-C1s antibody may be administered to a subject using any convenient means capable of resulting in the desired therapeutic effect or diagnostic effect. Thus, the anti-C1s antibody may be incorporated into a variety of formulations for therapeutic administration. For example, an anti-C1s antibody may be formulated into pharmaceutical compositions by combination with appropriate, pharmaceutically acceptable carriers, pharmaceutically acceptable diluents, or other pharmaceutically acceptable excipients and can be formulated into preparations in solid, semi solid, liquid or gaseous forms, such as tablets, capsules, powders, granules, ointments, solutions, suppositories, injections, inhalants and aerosols. In some embodiments, a pharmaceutical composition comprises an anti-C1s antibody and a pharmaceutically acceptable excipient.

In pharmaceutical dosage forms, an anti-C1s antibody can be administered in the form of their pharmaceutically acceptable salts, or they can also be used alone or in appropriate association, as well as in combination, with other pharmaceutically active compounds.

For oral preparations, an anti-C1s antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An anti-C1s antibody can be formulated into preparations for injection by dissolving, suspending or emulsifying the antibody in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, the pharmaceutical composition of the present disclosure can comprise further agents such as dopamine or psychopharmacologic drugs, depending on the intended use of the pharmaceutical composition.

Pharmaceutical compositions comprising an anti-C1s antibody are prepared by mixing a subject antibody having the desired degree of purity with optional physiologically acceptable carriers, other excipients, stabilizers, surfactants, buffers and/or tonicity agents. Acceptable carriers, other excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

Exemplary antibody concentrations in a pharmaceutical composition suitable for use in a method of the present disclosure can range from about 1 mg/mL to about 200 mg/mL or from about 50 mg/mL to about 200 mg/mL, or from about 150 mg/mL to about 200 mg/mL. In some aspects, the antibody concentration is from about 10 mg/mL to about 60 mg/mL, from about 12 mg/mL to about 58 mg/mL, from about 14 mg/mL to about 56 mg/mL, from about 16 mg/mL to about 54 mg/mL, from about 17 mg/mL to about 52 mg/mL, or from about 18 mg/mL to about 50 mg/mL. In some aspects, the antibody concentration is 18 mg/mL. In some aspects, the antibody concentration is 50 mg/mL.

An aqueous formulation of an anti-C1s antibody can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark TWEEN 80™). Examples of suitable polyethylene-polypropylene copolymers are those sold under the names PLURONIC® F68 or POLOXAMER 188™. Examples of suitable Polyoxyethylene alkyl ethers are those sold under the trademark BRIJ™. Exemplary concentrations of surfactant can range from about 0.001% to about 1% w/v.

A lyoprotectant can also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a suitable formulation includes an anti-C1s antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a suitable formulation can be a liquid or lyophilized formulation suitable for parenteral administration, and can comprise: about 1 mg/mL to about 200 mg/mL of a subject antibody; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a suitable parenteral formulation is a liquid or lyophilized formulation comprising: about 1 mg/mL to about 200 mg/mL of an anti-C1s antibody; 0.04% Tween 20 w/v; 20 mM L-histidine; and 250 mM sucrose; and has a pH of 5.5.

As another example, a subject parenteral formulation comprises a lyophilized formulation comprising: 1) 15 mg/mL of an anti-C1s antibody; 0.04% Tween 20 w/v; 20 mM L-histidine; and 250 mM sucrose; and has a pH of 5.5; or 2) 75 mg/mL of a subject antibody; 0.04% Tween 20 w/v; 20 mM L-histidine; and 250 mM sucrose; and has a pH of 5.5; or 3) 75 mg/mL of an anti-C1s antibody; 0.02% Tween 20 w/v; 20 mM L-histidine; and 250 mM sucrose; and has a pH of 5.5; or 4) 75 mg/mL of an anti-C1s antibody; 0.04% Tween 20 w/v; 20 mM L-histidine; and 250 mM trehalose; and has a pH of 5.5; or 5) 75 mg/mL of an anti-C1s antibody; 0.02% Tween 20 w/v; 20 mM L-histidine; and 250 mM trehalose; and has a pH of 5.5.

As another example, a suitable parenteral formulation is a liquid formulation comprising: 1) 7.5 mg/mL of an anti-C1s antibody; 0.02% Tween 20 w/v; 120 mM L-histidine; and 250 125 mM sucrose; and has a pH of 5.5; or 2) 37.5 mg/mL of an anti-C1s antibody; 0.02% Tween 20 w/v; 10 mM L-histidine; and 125 mM sucrose; and has a pH of 5.5; or 3) 37.5 mg/mL of an anti-C1s antibody; 0.01% Tween 20 w/v; 10 mM L-histidine; and 125 mM sucrose; and has a pH of 5.5; or 4) 37.5 mg/mL of an anti-C1s antibody; 0.02% Tween 20 w/v; 10 mM L-histidine; 125 mM trehalose; and has a pH of 5.5; or 5) 37.5 mg/mL of an anti-C1s antibody; 0.01% Tween 20 w/v; 10 mM L-histidine; and 125 mM trehalose; and has a pH of 5.5; or 6) 5 mg/mL of an anti-C1s antibody; 0.02% Tween 20 w/v; 20 mM L-histidine; and 250 mM trehalose; and has a pH of 5.5; or 7) 75 mg/mL of an anti-C1s antibody; 0.02% Tween 20 w/v; 20 mM L-histidine; and 250 mM mannitol; and has a pH of 5.5; or 8) 75 mg/mL of an anti-C1s antibody; 0.02% Tween 20 w/v; 20 mM L histidine; and 140 mM sodium chloride; and has a pH of 5.5; or 9) 150 mg/mL of an anti-C1s antibody; 0.02% Tween 20 w/v; 20 mM L-histidine; and 250 mM trehalose; and has a pH of 5.5; or 10) 150 mg/mL of an anti-C1s antibody; 0.02% Tween 20 w/v; 20 mM L-histidine; and 250 mM mannitol; and has a pH of 5.5; or 11) 150 mg/mL of an anti-C1s antibody; 0.02% Tween 20 w/v; 20 mM L-histidine; and 140 mM sodium chloride; and has a pH of 5.5; or 12) 10 mg/mL of an anti-C1s antibody; 0.01% Tween 20 w/v; 20 mM L-histidine; and 40 mM sodium chloride; and has a pH of 5.5.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985. The composition or formulation to be administered will, in any event, contain a quantity of a subject antibody adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

The present disclosure provides a method of treating a complement-mediated disease in an individual, the method comprising administering an anti-C1s antibody to the individual, where the anti-C1s antibody is administered in an effective amount of at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, or at least 10 g.

In some embodiments, the anti-C1s antibody is administered in an effective amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some embodiments, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some embodiments, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

In one aspect, the present disclosure provides a method of treating a complement-mediated disease in an individual, the method comprising administering an anti-C1s antibody to the individual, where the anti-C1s antibody is administered in an amount of 5.5 g. In some embodiments, a dose of 5.5 g of the anti-C1s antibody is administered to the individual every other week. In some embodiments, the method comprises: a) administering 5.5 g of the anti-C1s antibody on Day 1; b) administering 5.5 g of the anti-C1s antibody on Day 8; and c) administering 5.5 g of the anti-C1s antibody every other week following the Day 8 administration. In some embodiments, a dose of 5.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some embodiments, a dose of 5.5 g of the anti-C1s antibody is administered to the individual every other week for a period of time of more than 1 year. For example, in some embodiments, a dose of 5.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In some embodiments, the individual for the present method weighs 75 kg or more and the anti-C1s antibody is administered at an effective dose of about 7.5 g. In other aspects, the individual for the present method weighs less than 75 kg and the anti-C1s antibody is administered at an effective dose of about 6.5 g.

In another aspect, the present disclosure also provides a method of treating a complement-mediated disease in an individual, the method comprising administering an anti-C1s antibody to the individual, where the anti-C1s antibody is administered in an effective dose of about 6.5 g. In some embodiments, an effective dose of about 6.5 g of the anti-C1s antibody is administered to the individual every other week. In some embodiments, the method comprises: a) administering an effective dose of about 6.5 g of the anti-C1s antibody on Day 1; b) administering an effective dose of about 6.5 g of the anti-C1s antibody on Day 8; and c) administering an effective dose of about 6.5 g of the anti-C1s antibody every other week following the Day 8 administration. In some embodiments, an effective dose of about 6.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some embodiments, an effective dose of about 6.5 g of the anti-C1s antibody is administered to the individual every other week for a period of time of more than 1 year. For example, in some embodiments, an effective dose of about 6.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In another aspect, the present disclosure also provides a method of treating a complement-mediated disease in an individual, the method comprising administering an anti-C1s antibody to the individual, where the anti-C1s antibody is administered in an effective dose of about 7.5 g. In some embodiments, an effective dose of about 7.5 g of the anti-C1s antibody is administered to the individual every other week. In some embodiments, the method comprises: a) administering an effective dose of about 7.5 g of the anti-C1s antibody on Day 1; b) administering an effective dose of about 7.5 g of the anti-C1s antibody on Day 8; and c) administering an effective dose of about 7.5 g of the anti-C1s antibody every other week following the Day 8 administration. In some embodiments, an effective dose of about 7.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some embodiments, an effective dose of about 7.5 g of the anti-C1s antibody is administered to the individual every other week for a period of time of more than 1 year. For example, in some embodiments, an effective dose of about 7.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In other aspects, the present disclosure provides a method of treating a complement-mediated disease in an individual, the method comprising administering an anti-C1s antibody to the individual, where the anti-C1s antibody is administered in an effective dose between about 6.5 g and about 7.5 g. In some embodiments, an effective dose between about 6.5 g to about 7.5 g of the anti-C1s antibody is administered to the individual every other week. In some embodiments, the method comprises administering an effective dose between about 6.5 g and about 7.5 g of the anti-C1s antibody on Days 0 and 7 and then every other week thereafter. In some embodiments, an effective dose between about 6.5 g and 7.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some embodiments, an effective dose between about 6.5 g and 7.5 g of the anti-C1s antibody is administered to the individual every other week for a period of time of more than 1 year.

The present disclosure provides a method of treating a complement-mediated disease in a subject in need thereof, the method comprising administering an effective dose of an anti-C1s antibody to the subject, where the serum concentration of the anti-C1s antibody after the administration is at least about 20 μg/mL, at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, or at least about 100 μg/mL. In some embodiments of the disclosure, the serum concentration of the anti-C1s antibody after the administration is between about 20 μg/mL and about 100 μg/mL, about 20 μg/mL and about 90 μg/mL, about 20 μg/mL and about 80 μg/mL, about 20 μg/mL and about 70 μg/mL, about 20 μg/mL and about 70 μg/mL, about 20 μg/mL and about 60 μg/mL, about 20 μg/mL and about 50 μg/mL, about 20 μg/mL and about 40 μg/mL, or about 20 μg/mL and about 30 μg/mL. In some embodiments, the serum concentration of the anti-C1s antibody after the administration is at least about 20 μg/mL.

The serum concentration of the anti-C1s antibody in the subject can be measured using techniques known in the art. In some embodiments, the anti-C1s antibody is measured using a direct binding Enzyme-Linked Immunosorbent Assay (ELISA). In some embodiments, the anti-C1s antibody is measured using an indirect ELISA. In some embodiments, the anti-C1s antibody is measured using a sandwich ELISA. In some embodiments the anti-C1s antibody is measured using a competitive ELISA.

The present disclosure provides a method of treating a complement-mediated disease in a subject in need thereof, the method comprising administering an effective dose of an anti-C1s antibody to the subject, wherein the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg. In some embodiments, the effective dose of the anti-C1s antibody is at least about 60 mg/kg.

In some embodiments, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some embodiments, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some embodiments, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some embodiments, the effective dose for the present methods is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

The present disclosure provides a method of treating a complement-mediated disease in a subject in need thereof, the method comprising administering an effective dose of an anti-C1s antibody to the subject, wherein the anti-C1s antibody is administered at a dosing interval of five days, six days, seven days, eight days, nine days, ten days, eleven days, twelve days, thirteen days, fourteen days, fifteen days, sixteen days, seventeen days, eighteen days, nineteen days, twenty days, twenty one days, twenty two days, twenty three days, twenty four days, twenty five days, twenty six days, twenty seven days, twenty eight days, twenty nine days, thirty days, or thirty one days.

In some embodiments, the anti-C1s antibody is administered at a dosing interval of one week, two weeks, three weeks, four weeks, one month, two months, three months, or four months. In some embodiments, the anti-C1s antibody increases the number of reticulocytes in the subject's blood after the administration of the anti-C1s antibody.

In some embodiments, the anti-C1s antibody is administered as one or more loading doses followed by dosing at dosing intervals. The loading doses can be administered about 7 days apart, about 14 days apart, about 21 days apart, about 28 days apart, about two months apart, about three months apart, or about four months apart. In some embodiments, the loading dose for the present disclosure is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg. In some embodiments, the loading dose is a different dosage amount than the dose administered at dosing intervals. In some embodiments, the loading dose is the same dosage amount as the dose administered at dosing intervals. In one aspect, the anti-C1s antibody is administered as two weekly loading doses of 60 mg/kg followed by doses of 60 mg/kg administered every other week.

### Route of Administration

An anti-C1s antibody is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intrathecal, intracranial, subcutaneous, intradermal, topical, intravenous, intraperitoneal, intraarterial (e.g., via the carotid artery), spinal or brain delivery, rectal, nasal, oral, and other enteral and parenteral routes of administration. Routes of administration can be combined, if desired, or adjusted depending upon the antibody and/or the desired effect. An anti-C1s antibody composition can be administered in a single dose or in multiple doses. In some embodiments, an anti-C1s antibody is administered orally. In some embodiments, an anti-C1s antibody is administered subcutaneously. In some embodiments, an anti-C1s antibody is administered intramuscularly. In some embodiments, an anti-C1s antibody is administered intravenously.

An anti-C1s antibody can be administered to a host using any available conventional methods and routes suitable for delivery of conventional drugs, including systemic or localized routes. In general, routes of administration contemplated by the disclosure include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

By “treatment” is meant at least an amelioration of the symptoms associated with the pathological condition afflicting the host, where amelioration is used in a broad sense to refer to at least a reduction in the magnitude of a parameter, e.g., symptom, associated with the pathological condition being treated, such as a complement-mediated disease. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

In some embodiments, an anti-C1s antibody is administered by injection and/or delivery, e.g., to a site in a brain artery or directly into brain tissue. An anti-C1s antibody can also be administered directly to a target site e.g., by biolistic delivery to the target site.

A variety of hosts (wherein the term “host” is used interchangeably herein with the terms “subject,” “individual,” and “patient”) are treatable according to the subject methods. Generally such hosts are “mammals” or “mammalian,” where these terms are used broadly to describe organisms which are within the class mammalia, including the orders carnivore (e.g., cats), herbivores (e.g., cattle, horses, and sheep), omnivores (e.g., dogs, goats, and pigs), rodentia (e.g., mice, guinea pigs, and rats), and primates (e.g., humans, chimpanzees, and monkeys). In some embodiments, the host is an individual that has a complement system, such as a mammal, fish, or invertebrate. In some embodiments, the host is a complement system-containing mammal, fish, or invertebrate companion animal, agricultural animal, work animal, zoo animal, or lab animal. In some embodiments, the individual is human.

### Complement-Mediated Diseases

In some embodiments, a complement-mediated disease is characterized by the presence in a cell, a tissue, or a fluid of an elevated (higher than normal) amount of C1s or of an elevated level of complement C1s activity. For example, in some embodiments, a complement-mediated disease is characterized by the presence in brain tissue and/or cerebrospinal fluid of an elevated amount and/or an elevated activity of C1s. A “higher than normal” amount of C1s in a cell, a tissue, or a fluid indicates that the amount of C1s in the cell, tissue or fluid is higher than a normal, control level, e.g., higher than a normal, control level for an individual or population of individuals of the same age group. A “higher than normal” level of C1s activity in a cell, a tissue, or a fluid indicates that the proteolytic cleavage effected by C1s in the cell, tissue or fluid is higher than a normal, control level, e.g., higher than a normal, control level for an individual or population of individuals of the same age group. In some embodiments, an individual having a complement-mediated disease exhibits one or more additional symptoms of such a disease. It should be understood that the term “disease” encompasses “disorders.” The two terms may be used interchangeably. In some embodiments, a complement-mediated disease is a classical complement-mediated disease.

In some embodiments, a complement-mediated disease is characterized by the presence in a cell, a tissue, or a fluid of a lower than normal amount of C1s or of a lower level of complement C1s activity. For example, in some embodiments, a complement-mediated disease is characterized by the presence in brain tissue and/or cerebrospinal fluid of a lower amount and/or a lower activity of C1s. A “lower than normal” amount of C1s in a cell, a tissue, or a fluid indicates that the amount of C1s in the cell, tissue or fluid is lower than a normal, control level, e.g., lower than a normal, control level for an individual or population of individuals of the same age group. A “lower than normal” level of C1s activity in a cell, a tissue, or a fluid indicates that the proteolytic cleavage effected by C1s in the cell, tissue or fluid is lower than a normal, control level, e.g., lower than a normal, control level for an individual or population of individuals of the same age group. In some embodiments, an individual having a complement-mediated disease exhibits one or more additional symptoms of such a disease.

A complement-mediated disease is a disease in which the amount or activity of complement C1s is such as to cause disease in an individual. Non-limiting examples of complement-mediated diseases include cold agglutinin disease (CAD), bullous pemphigoid, multifocal motor neuropathy (MMN), autoantibody mediated peripheral neuropathy, Myasthenia Gravis, lupus nephritis, mucous membrane pemphigoid, cicatricial pemphigoid, ocular pemphigoid, and antineutrophil cytoplasmic autoantibody (ANCA) associated vasculitis.

In some embodiments, the present method includes treatment of primary CAD in a subject in need thereof comprising administering an effective dose between about 6.5 g and about 7.5 g, e.g., about 6.5 g for subjects with less than 75 kg of bodyweight and 7.5 g for subjects with 75 kg or more of bodyweight, of an anti-C1s antibody, e.g., sutimlimab. In some embodiments, the present methods have no limitation of use associated with anemia severity, transfusion history, or prior treatment experience. In some embodiments, there is no REMS requirement prior to dosing; vaccinate patients according to local guidelines prior to treatment initiation to reduce risk of serious infection. In some embodiments, the dose is administered as intravenous infusion over 1 hour on Day 0, Day 7, and every 14 days±2 days thereafter starting on Day 21. Intravenous infusion can take place within clinic or home setting. As a result of the treatment, the anti-C1s antibody can improve anemia and associated clinical symptoms, eliminate transfusion, prevent hemolysis, rapid onset of action, improve fatigue and quality of life, and/or any combination thereof. In other embodiments, the treatment shows no drug related serious or severe adverse events; no discontinuations due to adverse events, no serious infections; no REMS requirement, most commonly reported adverse events were similar to placebo, or any combination thereof. In other embodiments, as a result of the treatment, the anti-C1s antibody prevents chronic hemolysis, resulting in improvement in anemia, elimination of transfusion, improvement of quality of life, and ultimately reduction of risk of life-threatening thromboembolic events, morbidity, and mortality, and reduced healthcare utilization. In some embodiments, the anti-C1s antibody improves fatigue.

In some embodiments, the complement-mediated disease is bullous pemphigoid. In some embodiments, the complement-mediated disease is antibody-mediated rejection of organ transplant. In some embodiments, the complement-mediated disease is cold agglutinin disease. In some embodiments, the complement-mediated disease is warm autoimmune hemolytic anemia. In some embodiments, the complement-mediated disease antibody-mediated transplant rejection. In some embodiments, the classical complement-mediated disease is immunothrombocytopenic purpura. In some embodiments, the complement-mediated disease is neuromyelitis optica.

In some embodiments, the complement-mediated disease is multifocal motor neuropathy (MMN). In some embodiments, the complement-mediated disease is myasthenia gravis. In some embodiments, the complement-mediated disease is chronic inflammatory demyelinating polyneuropathy. In some embodiments, the complement-mediated disease is lupus nephritis. In some embodiments, the complement-mediated disease is mucous membrane pemphigoid. In some embodiments, the complement-mediated disease is cicatricial pemphigoid. In some embodiments, the complement-mediated disease is ocular pemphigoid. In some embodiments, the complement-mediated disease is antineutrophil cytoplasmic autoantibody (ANCA) associated vasculitis.

In other embodiments, the complement-mediated disease is an autoantibody mediated peripheral neuropathy including, but not limited to, Guillain-Barré syndrome, Myasthenia Gravis, acute inflammatory demyelinating polyneuropathy (AIDP), chronic inflammatory demyelinating polyneuropathy (CIDP), acute motor axonal neuropathy (AMAN), acute motor and sensory axonal neuropathy (AMSAN), pharyngeal-cervical brachial variant, Miller Fisher syndrome, or any combination thereof. In some embodiments, the complement-mediated disease is Guillain-Barré syndrome, which presents as rapid-onset muscle weakness, beginning in the feet and hands that spreads to the arms and upper body. During the acute phase, it can be fatal as respiratory failure can occur, and other autonomic functions (such as heart rate) can be affected. ˜7.5% of all cases are fatal. Incidence: 1-2/100,000.

In other embodiments, the complement-mediated disease is Myasthenia Gravis, which exhibits weakness, fatigue that becomes progressively worse during periods of physical activity, generally starts with ocular weakness; progressing to a more severe form, characterized by weakness in the extremities and performing basic life functions (chewing, swallowing, breathing). In a myasthenic crisis, respiratory paralysis occurs, necessitating assisted ventilation to sustain life.

In other embodiments, the complement-mediated disease is multifocal motor neuropathy (MMN), which is an inflammatory autoimmune disease of the lower nervous system. MMN is a pure motor neuropathy, which has the mean age onset of 40 years. MMN is characterized by: slowly progressive, asymmetric distal limb weakness; conduction block (CB), often affecting ulnar, median, radial or tibial nerves; and/or atrophic muscles. Other clinical features include muscle cramps, fasciculations, and an increase of weakness in cold conditions. GM1-specific IgM antibodies are present in the serum of ˜half of all patients, titers of which correlate with their in vitro complement-activating capacity and disease severity. Intravenous immunoglobulin (IVIg) is effective in MMN. Nevertheless, patients still undergo slowly progressive axonal degeneration and muscle weakness that cannot be fully prevented with chronic IVIg therapy.

In other embodiments, a complement-mediated disease useful for treatment is neuromyelitis optica (NMO). NMO is caused by anti-Aquaporin-4 IgG autoantibody (NMO-IgG) which activates complement and kills astrocytes resulting in death of oligodendrocytes that myelinate the optic nerve and spinal cord. Vision loss and paralysis occur following attacks.

In other embodiments, a complement-mediated disease useful for treatment is systemic lupus erythematosus (SLE). Systemic lupus erythematosus (SLE) is an autoimmune disease that affects 0.04% of the population of developed countries. SLE is believed to arise as a result of an impairment in the body's waste disposal system, in which complement plays a key role. In humans, congenital deficiencies of the complement proteins in the C1 complex as well as C2 and C4 are associated with an increased risk of developing SLE. However, a substantial number of patients with SLE develop hypocomplementemia with depletion of C1q and other components of the classical pathway: e.g., complement deposition on RBCs and/or C1q deposition in affected tissues.

In other embodiment, a complement-mediated disease useful for treatment is lupus nephritis (LN). LN is the renal manifestation of SLE that occurs in 25-50% of patients and is the primary cause of morbidity and mortality. C1q antibodies are closely associated with renal involvement and are highly predictive of and present during flares. Active LN is rarely observed in the absence of C1q Abs. Multiple studies have shown a negative correlation with C1q Ab titers and serum C1q, and a positive correlation with C1q deposition in the glomeruli in patients with LN.

In some embodiments, a complement-mediated disease useful for treatment is membranoproliferative glomerulonephritis (type I) (Mixed Cryoglobulinemia). Mixed Cryoglobulinemia is a systemic vasculitis mediated by immune complexes (IC). It appears most often in the context of chronic infections (HCV—80% of MC cases). Clinically, cryoglobulinemia manifests itself with symptoms like weakness and arthralgias and variable cutaneous and visceral organ involvement. Steroids suppress inflammation with success in some patients, but additional plasmapheresis to remove circulating cryoglobulins and immunosuppressive treatment to inhibit the formation of new cryoglobulins are often necessary.

### Examples

In a Phase 3, pivotal, open-label, multicenter study to assess the efficacy and safety of sutimlimab in patients with primary cold agglutinin disease (CAD) who have a recent history of blood transfusion (ClinicalTrials.gov Identifier: NCT03347396; EudraCT No. 2017-003538-10; EFC16215), patients with CAD received intravenous doses of sutimlimab on Days 0 and 7, and biweekly infusions thereafter. Patients weighing <75 kg received a 6.5 g dose, and those weighing ≥75 kg received a 7.5 g dose. Cytokine levels for IL-6 and IL-10 were assessed in patients with available serum samples. Cytokine profiles and functional assessment of chronic illness therapy-fatigue (FACIT-F) scores were evaluated from baseline to follow-up timepoints at weeks 1, 3, 5 and 25 as represented by the treatment assessment timepoint (TAT) post-sutimlimab treatment. Summary statistics describing IL-6 and IL-10 changes at each week were reported. Changes from baseline to the TAT were analyzed using the Mixed Model for Repeated Measures (MMRM). A 3- to 10-point score improvement in FACIT-F was considered a meaningful difference in patient fatigue (based on FACIT-F data in autoimmune or oncologic diseases; Lai et al. J Rheumatol. 2011 and Reddy et al. J Palliat Med. 2007).

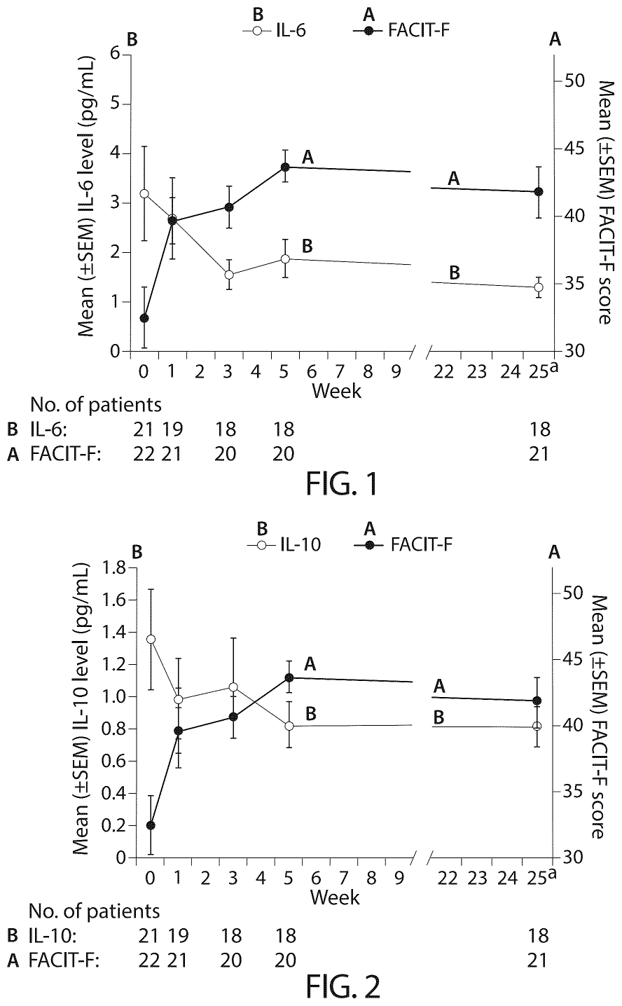

Mean IL-6 level (mean pg/mL [standard error of the mean (SEM)]) steadily decreased from baseline (3.21 [0.958]; normal values IL-6<3.2 pg/mL.) to follow up at all time points after initiating sutimlimab treatment (FIG. 1), showing rapid onset and durable pattern of decline as early as Week 1 (2.70 [0.839]). Mean IL-6 level was reduced by more than half by Week 3 (1.56 [0.297]), slightly rose at Week 5 (1.88 [0.383]) and was lowest at TAT (1.31 [0.201]). Mean IL-10 level (pg/mL [SEM]) also decreased in a time-dependent manner from baseline (1.36 [0.310]) initially at Week 1 (0.99 [0.250]). Mean IL-10 marginally increased at Week 3 (1.07 [0.306]) but dropped at Week 5 (0.83 [0.142]) and was lowest by TAT (0.82 [0.129]) with sutimlimab treatment (FIG. 2). At baseline, mean (SEM) FACIT-F score was 32.5 (2.265) (FIGS. 1-2). Patient FACIT-F scores showed early and late improvements with mean score increases at Week 1 (39.67 [1.740]), Week 3 (40.70 [1.542]), Week 5 (43.75 [1.191]), and at TAT (41.86 [1.958]). Decreased inflammation, as demonstrated by IL-6 and IL-10 activity, correlated inversely with FACIT-F score improvements over time.

In this Phase 3 study, decrease from baseline to TAT in proinflammatory cytokine IL-6 levels and regulatory cytokine IL-10 levels were observed during sutimlimab treatment, highlighting the influence of complement inhibition on inflammation in CAD. Improved FACIT-F scores are concurrent with sutimlimab treatment and inhibition of the classical complement pathway. An inverse correlation was noted between select inflammatory cytokines and meaningful improvements in patients' fatigue, suggesting complement-mediated inflammation may additionally contribute to manifestation of fatigue in patients with CAD.

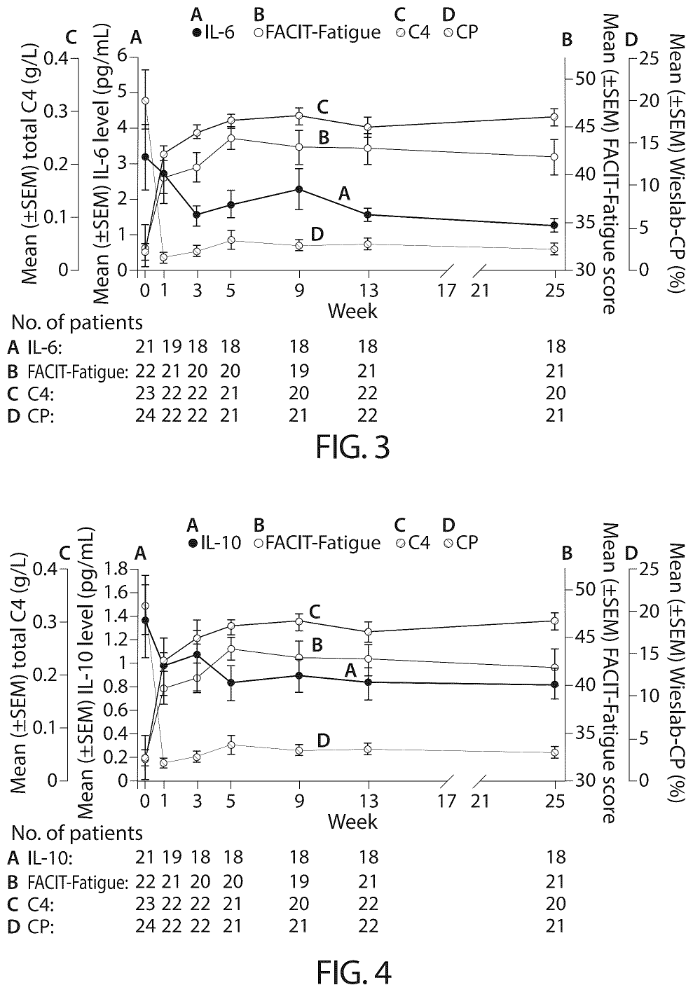

Additional time points and parameters are presented in FIGS. 3 and 4. Cytokine profiles for IL-6 and IL-10, FACIT-F scores, and hemoglobin (Hb) levels were evaluated from baseline to Weeks 1, 3, 5, 9, 13, and 25 after the first sutimlimab dose (Week 25 represented the treatment assessment time point [TAT]). FACIT-F is a patient-reported quality of life (QOL) outcome that is a validated assessment tool for measuring fatigue in patients with CAD (Roth A et al. N Engl J Med. 384(14):1323-1334 (2021) and Hill Q A et al. EHA 2021; Poster). FACIT-Fatigue scale range is 0 (worst fatigue) to 52 (no fatigue); a 3-point increase from baseline is considered a clinically meaningful improvement (Röth A et al. N Engl J Med. 384(14):1323-1334 (2021)). Activity of the classical complement pathway was measured via the Wieslab CP assay. Descriptive summary statistics of changes in IL-6, IL-10, FACIT-Fatigue, Hb, C4 and classical complement pathway activity were reported at each time point.

Mean (SEM) IL-6 level was lower than baseline (3.21 [0.958] pg/mL) as early as Week 1 (2.70 [0.839] pg/mL) and at all other timepoints after initiating sutimlimab treatment (FIG. 3). Compared to baseline, mean IL-6 level decreased by more than half at Week 3 (1.56 [0.297] pg/mL), a level that was maintained at Week 13 (1.57 [0.201] pg/mL), and lower still at the TAT (1.31 [0.201] pg/mL). Mean (SEM) IL-10 level also declined from baseline (1.36 [0.310] pg/mL) to follow-up during sutimlimab treatment (FIG. 4), with a reduction observed as early as Week 1 (0.99 [0.250] pg/mL). Mean (SEM) IL-10 was 0.83 (0.132) pg/mL at Week 13 and was lowest at the TAT (0.82 [0.129] pg/mL). Decreased levels of IL-6 and IL-10 coincided with rapid and durable increases in mean FACIT-Fatigue score (i.e., decreased fatigue). At baseline, mean (SEM) FACIT-Fatigue score was 32.5 (2.3), consistent with the level of fatigue reported for patients with paroxysmal nocturnal hemoglobinuria (Schrezenmeier H et al. Haematologica 99(5):922-929 (2014)) and cancer (Escalante C P et al. Cancer Med. 8(2):543-553 (2019)). Clinically meaningful improvements in fatigue were observed at Week 1 (7-point mean score improvement) and TAT (10-point mean score improvement) and coincided with inhibition of the classical complement pathway. Sutimlimab treatment led to rapid suppression of Wieslab CP activity and normalization of mean total C4 level, effects that were maintained over the treatment period.

Thus, treatment with sutimlimab, a selective C1s inhibitor, was associated with rapid and durable decreases in inflammatory cytokines (IL-6, IL-10) from baseline to follow up at the TAT, highlighting the effects of classical complement pathway inhibition (i.e., suppression of Wieslab CP activity and normalized total C4 level) in patients with CAD. Concurrent inverse changes over time were observed for these select inflammatory/regulatory cytokines and fatigue; these results suggest that, in addition to anemia, complement-mediated inflammation may contribute to fatigue in patients with CAD and further support C1s inhibition as an effective therapeutic target for this disorder.

1. A method comprising administering to a subject an anti-C1s antibody; measuring a level of IL-6, and/or IL-10 in a sample from the subject; and optionally assessing fatigue in the subject.

2. (canceled)

3. The method of claim 1, wherein the subject has a complement-mediated disease, optionally a classical complement-mediated disease, further optionally cold agglutinin disease (CAD).

4. The method of claim 1, wherein the subject has fatigue.

5. A method comprising treating a subject with an anti-C1s antibody, wherein the subject has fatigue; measuring a level of IL-6, and/or IL-10 in a sample from the subject; and optionally assessing fatigue in the subject.

6. The method of claim 5, wherein the subject has a complement-mediated disease, optionally a classical complement-mediated disease, further optionally cold agglutinin disease (CAD).

7. A method comprising treating a subject with an anti-C1s antibody, wherein the subject has a complement mediated disease, optionally cold agglutinin disease (CAD); measuring a level of IL-6, and/or IL-10 in a sample from the subject; and optionally assessing fatigue in the subject.

8. The method of claim 7, wherein the subject has fatigue.

9. The method of claim 1, wherein the subject has a baseline level of IL-6, and/or IL-10 prior to treatment with the anti-C1s antibody, and/or wherein the subject has a baseline level of fatigue prior to treatment with the anti-C1s antibody.

10. The method of claim 9, wherein:
(a) if the level of IL-6, and/or IL-10 in the sample is reduced, optionally by at least 10%, relative to baseline and/or fatigue in the subject is improved relative to baseline, the method further comprises continuing with the current anti-C1s antibody treatment; or
(b) if the level of IL-6, and/or IL-10 in the sample is within 10% of baseline and/or fatigue the subject is maintained or worsens relative to baseline, the method further comprises altering the current anti-C1s antibody treatment.

11. The method of claim 10, wherein altering the current anti-C1s antibody treatment comprises: (i) adjusting the dosage and/or frequency of the treatment with the anti-C1s antibody, (ii) further treatment of the subject with an anti-inflammatory agent, or (iii) further treatment of the subject to improve fatigue.

12.-13. (canceled)

14. The method of claim 1, further comprising monitoring levels of IL-6, and/or IL-10 in the subject over a period of time.

15. The method of claim 1, wherein the subject has undergone a blood transfusion.

16. The method of claim 1, wherein the fatigue is assessed based on a Functional Assessment of Chronic Illness Therapy-Fatigue (FACIT-F) score, optionally wherein an improvement in fatigue is a change by at least 3 points on a FACIT-F score relative to baseline.

17. The method of claim 1, wherein the anti-C1s antibody comprises a heavy chain (HC) complementarity determining region 1 (CDR1) comprising the amino acid sequence of SEQ ID NO: 5, an HC complementarity determining region 2 (CDR2) comprising the amino acid sequence of SEQ ID NO: 5 6, an HC complementarity determining region 3 (CDR3) comprising the amino acid sequence of SEQ ID NO: 7, a light chain (LC) CDR1 that comprises the amino acid sequence of SEQ ID NO: 8, an LC CDR2 comprising the amino acid sequence of SEQ ID NO: 9, and an LC CDR3 comprising the amino acid sequence of SEQ ID NO: 10.

18. The method of claim 17, wherein the anti-C1s antibody comprises a heavy chain variable region (VH) comprising the amino acid sequence of SEQ ID NO: 3 and comprises a light chain variable region (VL) comprising the amino acid sequence of SEQ ID NO: 4.

19. The method of claim 18, wherein the anti-C1s antibody comprises an HC comprising the amino acid sequence of SEQ ID NO: 1 and an LC comprising the amino acid sequence of SEQ ID NO: 2.

20. The method of claim 1, wherein the anti-C1s antibody comprises a heavy chain (HC) complementarity determining region 1 (CDR1) comprising the amino acid sequence of SEQ ID NO: 15, an HC CDR2 comprising the amino acid sequence of SEQ ID NO: 16, an HC CDR3 comprising the amino acid sequence of SEQ ID NO: 17, a light chain (LC) complementarity determining region 1 (CDR1) comprising the amino acid sequence of SEQ ID NO: 18, an LC CDR2 comprising the amino acid sequence of SEQ ID NO: 19, and an LC CDR3 comprising the amino acid sequence of SEQ ID NO: 20.

21. The method of claim 20, wherein the anti-C1s antibody comprises a heavy chain variable region (VH) comprising the amino acid sequence of SEQ ID NO: 13 and comprises a light chain variable region (VL) comprising the amino acid sequence of SEQ ID NO: 14.

22. The method of claim 21, wherein the anti-C1s antibody comprises an HC comprising the amino acid sequence of SEQ ID NO: 11 and an LC comprising the amino acid sequence of SEQ ID NO: 12.

23. The method of claim 1, wherein the anti-C1s antibody comprises an IgG4 constant region.


SEQUENCE LISTING (24 sequences)

SEQ ID NO: 1 - protein, 445 aa, synthetic construct
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 216 aa, synthetic construct
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, synthetic construct
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSS

SEQ ID NO: 4 - protein, 109 aa, synthetic construct
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 5 - protein, 5 aa, synthetic construct
NYAMS

SEQ ID NO: 6 - protein, 17 aa, synthetic construct
TISSGGSHTYYLDSVKG

SEQ ID NO: 7 - protein, 9 aa, synthetic construct
LFTGYAMDY

SEQ ID NO: 8 - protein, 12 aa, synthetic construct
TASSSVSSSYLH

SEQ ID NO: 9 - protein, 7 aa, synthetic construct
STSNLAS

SEQ ID NO: 10 - protein, 10 aa, synthetic construct
HQYYRLPPIT

SEQ ID NO: 11 - protein, 446 aa, synthetic construct
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 12 - protein, 218 aa, synthetic construct
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSS

SEQ ID NO: 14 - protein, 111 aa, synthetic construct
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIK

SEQ ID NO: 15 - protein, 5 aa, synthetic construct
DDYIH

SEQ ID NO: 16 - protein, 17 aa, synthetic construct
RIDPADGHTKYAPKFQV

SEQ ID NO: 17 - protein, 10 aa, synthetic construct
YGYGREVFDY

SEQ ID NO: 18 - protein, 15 aa, synthetic construct
KASQSVDYDGDSYMN

SEQ ID NO: 19 - protein, 7 aa, synthetic construct
DASNLES

SEQ ID NO: 20 - protein, 9 aa, synthetic construct
QQSNEDPWT

SEQ ID NO: 21 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 24 - protein, 107 aa, synthetic construct [sequence omitted]